(19) United States
(12) Patent Application Publication
(10) Pub. No.: US 2022/0379004 A1
(43) Pub. Date: Dec. 1, 2022

(54) DRUG ASSISTED WOUND DRAINAGE LINE

(71) Applicant: Tennessee Technological University, Cookeville, TN (US)

(72) Inventors: Ann Hellman, Cookeville, TN (US); Mary Forde, Cookeville, TN (US); Pressley May, Cookeville, TN (US); David Matei, Gallatin, TN (US); Alexander Otts, Leeds, AL (US); Robert Sanders, Cookeville, TN (US); Sheila Hurley, Cookeville, TN (US); Nathan Beaty, Cookeville, TN (US)

(21) Appl. No.: 17/331,278

(22) Filed: May 26, 2021

(51) Int. Cl. A61M 1/00; A61M 5/14; A61M 25/00

(52) U.S. Cl. CPC A61M 1/92; A61M 1/90; A61M 5/14; A61M 25/003; A61M 25/0043; A61M 25/0075; A61M 2205/8206; A61M 2025/0004; A61M 2025/0031; A61M 2025/0057; A61M 2025/0076

(57) ABSTRACT

The wound drainage apparatus for drug-assisted removal of bodily fluids includes a double-lumen tubing member connected to a customizable branched tubing member for placement in a wound cavity. A perforated inner tube connected to an anticoagulant drug delivery pump and a solid outer tube connected to a fluid drainage vacuum make up the double-lumen tubing member. The branched tubing member includes a plurality of secondary wound cavity tubes branching away from a primary wound cavity tube. The apparatus disclosed herein improves upon current drainage systems by preventing occlusions produced by blood clots forming in fluid drainage tubes.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### CROSS-REFERENCES TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING OR COMPUTER PROGRAM LISTING APPENDIX

Not Applicable

### BACKGROUND

The present disclosure relates generally to wound drainage lines and bodily fluid removal during and after surgical procedures.

Wound draining lines and vacuum assisted closure (VAC) systems are generally known in the art. Wound drainage lines and VAC systems may be used in a variety of settings in which various bodily fluids need to be removed from a living organism's body (human, animal) during or after a surgical procedure. One such scenario includes a patient who has just undergone general surgery and now requires post-operative drainage of bodily fluids and blood from the surgical site.

In current wound vacuum assisted (VAC) systems, excess bodily fluid removal is accomplished by means of a suction pump in active communication with a drainage tube that extends into a patient's body cavity. Further, in current wound VAC systems, the drainage tube is a simple two-part component. During surgery, the surgeon inserts a porous tube into the body cavity, which provides a pathway for the removal of bodily fluid produced during the healing process. The porous portion terminates into a nonporous tube when exiting the body cavity. To properly secure the tube, the surgeon will suture or tape the tube to the patient. A flexible, 4 to 6 foot tube is then secured to the tube exiting the body to provide additional length for attachment to the vacuum source. Fluid is removed from the body cavity via a negative pressure gradient produced by a vacuum source.

Current drainage lines, however, do not have the ability to limit blood coagulation in the tubes, and proper drainage and fluid removal is often impeded due to the formation of blood clots inside the drainage tubing as well as on the exterior of the tubing inside the body cavity. This problem is typically solved by “milking” the stagnant tubing lines to break up the clots, which can be painful to patients. Thus, a wound drainage system that could prevent blood clots and occlusions from occurring in the tubing lines would greatly improve quality of patient care and could decrease the amount of time spent in the hospital during recovery. The elimination of stagnant tubing reduces the possibilities of bacterial growth and infections and can decrease time of healing, having an overall positive impact on patient and health care costs. Because removal of bodily fluid is crucial to patient comfort and health throughout the post-operative healing process, devising a practical, easy to use wound drainage systems capable of preventing occlusions and alleviating fouling produced by clotting would be of great benefit to patients and health care providers using such systems.

What is needed then are improvements in wound drainage systems and methods for drainage of bodily fluids from surgical sites.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

The apparatuses and methods described herein involve the integration of an anticoagulant drug delivery system with the primary drainage tube in a wound VAC system to alleviate and prevent dangerous clot formation. In some embodiments, the present disclosure provides a device for the drainage of bodily fluids from a wound. For example, the new drainage system can comprise a concentric tube drainage system with an inner and outer tube. The outer tube can provide a pathway for bodily fluid removal via a negative pressure gradient and the inner tube can be the primary pathway for drug delivery. For the inner tube, a positive pressure gradient produced by a battery-powered pump will provide the necessary force for drug delivery. Perforations strategically spaced along the length of the inner tube wall will allow for the drug to enter the bodily fluid pathway. In some embodiments, the drug will remain inside of the tubing network so that no drug enters the body cavity. In other embodiments, however, the drug could potentially leave the tubing network and enter the body cavity, if needed. The newly designed tubing may connect to a branched silicon tube that is placed and secured in a patient's body cavity by a surgeon. The branched tubing design is provided as a more effective alternative for the removal of fluid due to an increase in overall surface area. Further, the branched tubing network can allow for customization of tube sizing and fit to a patient's specific wound geometry to improve fluid drainage.

In one aspect, the apparatus may include a branched, perforated tubing network with open or closed ends that is customizable to a specific wound geometry by incorporating tubing of various sizes, geometries, and configurations. In some embodiments, thin structures may be provided between the branched tubing to facilitate the placement of the tubing inside of a wound. In some embodiments, the branched tubing network may be non-perforated (i.e., solid) with open ends, the tubing still being customizable to specific wound geometries. The branched design provides multiple transport pathways and an adjustable surface area to facilitate wound fluid drainage when the tubing network is connected to a vacuum or other negative pressure pumping system. In such embodiments, a double-lumen tubing drug delivery system may also be placed exterior to the wound domain. This double-lumen tubing may enable the distribution of drugs (e.g. anticoagulants) through the ablumenal space via a pump and subsequent transport of the drug into an inner tube portion via transport through a the inner tube's porous wall. The area between the inner and outer tube may provide a pathway for bodily fluid removal via a negative pressure gradient. Such a design may also be capable of connecting to the branched tubing networks described herein.

In an embodiment, the wound drainage apparatus disclosed herein may comprise a double-lumen primary tubing member in fluid connection with a branched tubing member. In some embodiments, the double-lumen tubing member may that act as drug delivery system and further comprise two concentric tubes, an inner and an outer tube. The inner tube may be perforated and connected to a battery-powered pump on one end and closed on the other end, while the outer tube may be solid-walled and connected to a vacuum or other negative pressure device on one end and open at the other end. In some embodiments, the branched tubing member may be connected to the open end of the solid walled outer tube and placed and secured in a wound or body cavity by a surgeon. The branched tubing member can be made of silicon in one embodiment, but may be made from many other materials or combinations of materials in other embodiments, such as polyethylene, polyvinyl chloride (PVC), and/or any other medical grade plastic or polymer. In some embodiments, the branched tubing can be produced in three different sizes—small, medium, and large—in order to provide patient customization. In such embodiments, the surgeon can easily cut the branched tubing to better-fit specific wound geometries. The connection between the outer tube and the branched tubing member should be airtight, so as to ensure the negative pressure from the vacuum is experienced throughout the branched tubing member. In some embodiments, the connection between the outer tube and branched tubing can comprise a one-way valve, so as to only allow fluids to flow away from the body cavity. The branched tubing member may further comprise a primary wound cavity tube and a plurality of secondary wound cavity tubes branching from the primary wound cavity tube. In some embodiments, the primary and secondary wound cavity tubes can be perforated and have either open or closed ends. In other embodiments, however, the primary and secondary wound cavity tubes can be solid-walled and have open ends. The perforations and open ends of the primary and secondary wound cavity tubes function to allow bodily fluids from the wound cavity to enter the tubing.

In embodiments where the double-lumen tubing member and the branched tubing member are connected, the vacuum should be operable to produce a negative pressure gradient when activated, and drain bodily fluids away from the would cavity via the branched tubing member and the double-lumen member. In these embodiments, the battery-powered pump should also be operable to produce a positive pressure gradient when activated, and deliver the drug (e.g., anticoagulant) to the solid outer tube via the plurality of perforations in the perforated inner tube. From there, the anticoagulant drug can mix with the blood and other bodily fluids evacuating the wound cavity via the solid outer tube and ensure proper fluid drainage by alleviating clots formation in the drainage tubes. In such embodiment, the drug does not enter any portion of the branched tubing member due to the negative pressure gradient present in the outer tube. In other embodiments, a one-way valve may be placed at the connection between the double-lumen tubing member and the branched tubing member to serve as additional protection against the potentially life-threatening situation of the anticoagulant entering a patients wound cavity and preventing the clotting of blood therein.

Another aspect of the disclosure may include a method of draining fluids from a wound. The method may comprise the steps of providing a double-lumen member comprising a perforated inner tube for drug delivery and a solid outer tube for fluid drainage, providing an anticoagulant drug, creating a fluid connection between a pump-side end of the perforated inner tube and a battery-powered pump that is operable to produce positive pressure gradient and supply the anticoagulant drug, and creating a fluid connection between a pump-side end of the solid outer tube and a vacuum that is operable to produce a negative pressure gradient and drain bodily fluids from inside the outer tube member. In some embodiments, the method may comprise the additional steps of providing a branched tubing member comprising a primary wound cavity tube and a plurality of secondary wound cavity tube members branching off of the primary wound cavity tube, wherein the primary and plurality of secondary wound cavity tube members are perforated such that fluids can enter the tube members through the perforations. The method may further comprise placing the branched tubing member inside of a patient's wound cavity and sealing the branched tubing member therein, and attaching the open wound-side end of the solid outer tube to an open pump-side end of the primary wound cavity tube such that an airtight seal is created between the two. In some embodiments, the method further comprises activating the vacuum to provide a negative pressure gradient and drain bodily fluids away from the patient wound cavity via the branched tubing member and the solid outer tube, and activating the battery-powered pump to produce positive pressure gradient and deliver the anticoagulant drug to the solid outer tube via the plurality of perforations in the perforated inner tube, such that the anticoagulant drug does not enter any portion of the branched tubing member.

Numerous other objects, advantages and features of the present disclosure will be readily apparent to those of skill in the art upon a review of the drawings and description of a preferred embodiment.

### BRIEF DESCRIPTION OF THE DRAWINGS

FIG. 1 is an overall plan view of an exemplary embodiment of a wound drainage apparatus for the drug-assisted removal of bodily fluids.

FIG. 2 is a cross-section view of the exemplary embodiment of a double-lumen tubing member of the wound drainage apparatus identified in FIG. 1.

FIGS. 3A & 3B are schematics of two embodiments of a branched tubing member of the wound drainage apparatus.

FIG. 4 depicts yet another embodiment of a double-lumen tubing member inside of a patient's wound cavity.

### DETAILED DESCRIPTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific apparatus and methods described herein. Such equivalents are considered to be within the scope of this invention and are covered by the claims.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

The wound drainage apparatus for drug-assisted removal of bodily fluids disclosed herein includes a double-lumen tubing member connected to a customizable branched tubing member for placement in a wound cavity. A perforated inner tube connected to an anticoagulant drug delivery pump and a solid outer tube connected to a fluid drainage vacuum make up the double-lumen tubing member. The branched tubing member includes a plurality of secondary wound cavity tubes branching away from a primary wound cavity tube. The apparatus disclosed herein improves upon current drainage systems by preventing occlusions produced by blood clots forming in fluid drainage tubes.

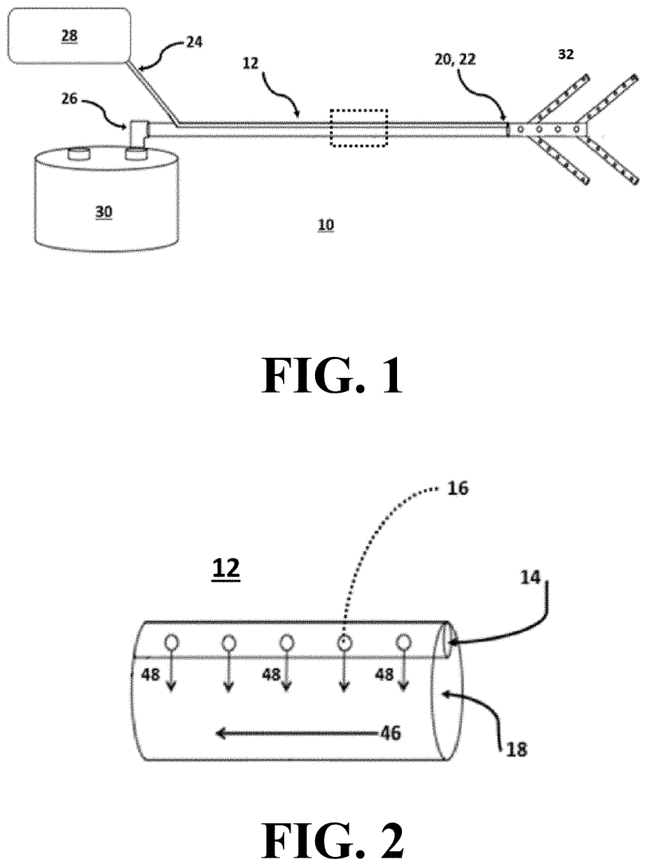

Referring now to FIGS. 1 and 2, an exemplary embodiment of a wound drainage apparatus 10 for the drug-assisted removal of bodily fluids generally comprises a double-lumen tubing member 12. The double-lumen tubing member 12 may further comprise a perforated inner tube 14 having a plurality of perforations 16 for drug delivery spaced throughout the tube 14 and a solid outer tube 18 for fluid drainage from a body cavity. In this embodiment, the perforated inner tube 14 and the solid outer tube 18 are substantially concentric tubes, with one being located inside of the other. While the perforated inner tube 14 and solid outer tube 18 have similarly located wound-side ends 20 and 22, respectively, they have separate and distinct pump-side ends. This is because each of their respective wound-side ends 20 and 22 must attach to different pressure devices in order for the wound drainage apparatus 10 to serve its intended purpose.

In the exemplary embodiment of apparatus 10 depicted in FIG. 1, the pump-side end of the perforated inner tube 24 is connected to a battery-powered pump 28. Battery-powered pump 28 is responsible for producing a positive pressure gradient throughout the internal length of perforated inner tube 14 and supplying a drug to the solid outer tube 18. The drug supply enters solid outer tube 18 after being forced in a flow direction 48 through the plurality of perforations 16 as a result of the positive pressure gradient produced by the battery-powered pump 28. While the supplied drug can be an anticoagulant like heparin in an exemplary embodiment, the supplied drug may be a number of other types of drug in other embodiments. Further, the pump-side end of the solid outer tube 26 is connected to a vacuum 30. In such an embodiment, the vacuum 30 is responsible for producing a negative pressure gradient throughout the internal length of the solid outer tube 18 and removing bodily fluids contained within the double-lumen tubing member 12. Due to the negative pressure gradient produced by vacuum 30, the flow direction of bodily fluids 46 contained within double-lumen tubing member 12 is a direction directly away from wound-side end 22.

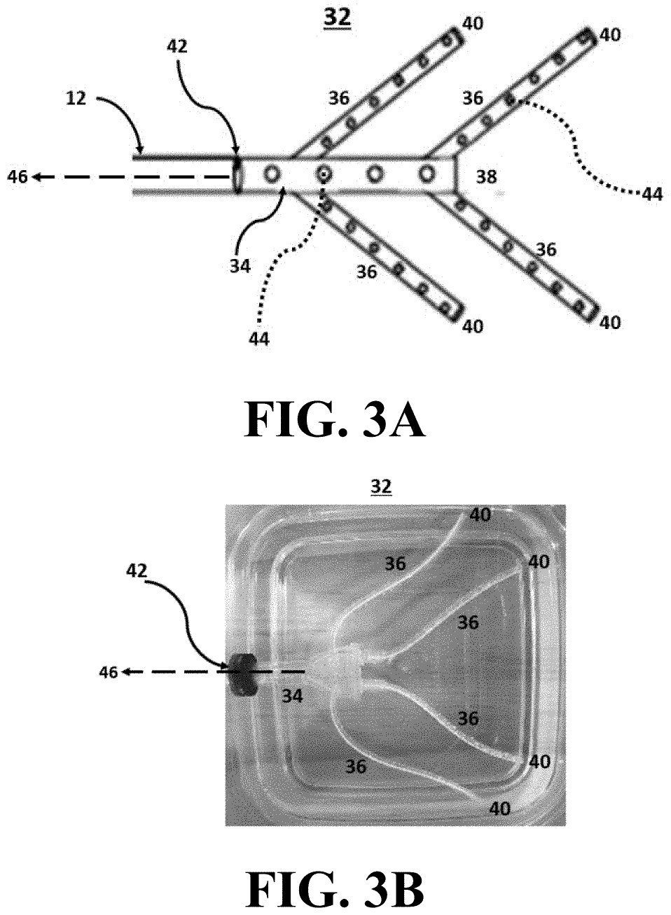

In a further embodiment of apparatus 10, the double-lumen tubing member 12 may be connected to a branched tubing member 32. As can be seen in FIGS. 3A and 3B, branched tubing member 32 may comprise a plurality of secondary wound cavity tube members 36 that branch from a primary wound cavity tube 42. Additional tube members can also branch off of the plurality of secondary wound cavity tube members 36 in certain embodiments. In some embodiments, primary and secondary wound cavity tubing members 34 and 36 may further comprise open or closed wound-side ends 38 and 40 in addition to a plurality of perforations 44. In other embodiments, the primary and secondary wound cavity tubing members 34 and 36 may comprise open wound-side ends 38 and 40 and contain no perforations (i.e. have solid walls). In these embodiments, the bodily fluids contained in a wound cavity 50 will enter primary and secondary wound cavity tube members 34 and 36 via their open wound-side ends 38 and 40 and/or the plurality of perforations 44. While the primary and plurality of secondary wound cavity tube members 34 and 36 may be separate and distinct pieces in an exemplary embodiment, they can be an integrally formed single piece in other embodiments. Further, the open wound-side end of solid outer tube 22 can be attached to an open pump-side end of the branched tubing member 42 in order to form an airtight fluid connection between the double-lumen tubing member 12 and branched tubing member 32. Once attached, the negative pressure gradient produced by vacuum 30 helps the removal of bodily fluids contained within the branched tubing member 32 in addition to those contained in double-lumen tubing member 12. In some embodiments, a one-way valve may be provided at the attachment point between members 12 and 32 so as to keep all fluids flowing in direction 46 and prevent the flow of any drug from reaching branched tubing member 32. The extra security a one-way valve may provide is desirable, as complications arising from anticoagulant drugs entering a recovering patient's surgical wound cavity can be potentially life-threatening. In other embodiments, the solid outer tube 18 of double-lumen tubing member 12, primary wound cavity tube 34, and the plurality of secondary cavity tube members 36 may be integrally formed.

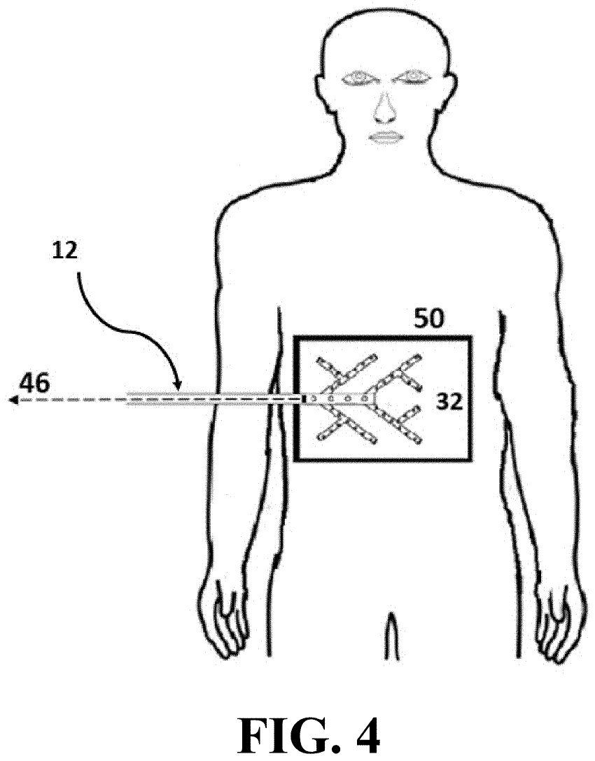

In an exemplary embodiment, such as that depicted in FIG. 4, the branched tubing member 32 of wound drainage apparatus 10 can be placed inside a patient's body or wound cavity 50 by a surgeon to aide in the proper drainage of fluids from cavity 50. The branched tubing's dimensions may be customizable on a patient-by-patient basis, in order to fit the unique geometry of a specific patient's wound cavity 50. For example, the branched tubing member 32 could be produced in three different sizes—small, medium, and/or large—and then subsequently cut by the surgeon to better fit a specific patient's wound dimensions. Thanks to its bifurcated design, the branched tubing member 32 has an increased surface area as compared to traditional single tube members, which allows for better fluid removal capability where the branched tubing member 32 is indirectly connected to the vacuum 30 or other negative pressure device via its direct connection with the wound-side end of the solid outer tube 22. While in some embodiments, the double-lumen tubing member 12 may extend to, but not enter, the patient wound cavity 50, the double-lumen tubing member 12 may enter wound cavity 50 in other embodiments.

In an exemplary embodiment where the branched tubing member 32 of the wound drainage apparatus 10 has been properly placed and secured in the patient's wound cavity 50, the activation of the vacuum 30 will initiate the drainage flow of bodily fluids, such as blood, in a direction 46 away from the wound cavity 50 and through both the branched tubing member 32 and the solid outer tube 18 of double-lumen tubing member 12. Furthermore, activation of battery-powered pump 28 will help to prevent blood clots from forming inside the double-lumen tubing member 12 by distributing an anticoagulant drug throughout the solid outer tube 18 via the plurality of perforations 16 located along the perforated inner tube 14. In this manner, wound drainage apparatus 10 improves the quality of patient care by alleviating the formation of dangerous blood clots and occlusions in wound drainage lines.

Thus, although there have been described particular embodiments of the present invention of a new and useful wound drainage apparatus, it is not intended that such references be construed as limitations upon the scope of this invention.

What is claimed is:

1. A wound drainage apparatus for the drug-assisted removal of bodily fluids comprising:
a double-lumen tubing member comprising:
a perforated inner tube having a plurality of perforations for drug delivery; and
a solid outer tube for fluid drainage;
wherein the perforated inner tube and the solid outer tube are substantially concentric, each having a similarly located wound-side end and a distinct pump-side end;
wherein the wound-side end of the perforated inner tube is closed and the pump-side end of the perforated inner tube is in fluid communication with a battery-powered pump that is operable to supply an anticoagulant drug through the perforated inner tube via a positive pressure gradient;
wherein the wound-side end of the solid outer tube is open, and the pump-side end of the solid outer tube is in fluid communication with a vacuum that is operable to remove bodily fluids via a negative pressure gradient;

2. The apparatus of claim 1, further comprising:
a branched tubing member for placement in a patient wound cavity comprising:
a primary wound cavity tube; and
a plurality of secondary wound cavity tube members branching off of the primary wound cavity tube;
wherein each of the primary would cavity tube member and plurality of secondary wound cavity tube members have a wound-side end and a pump-side end;
wherein the primary wound cavity tube and the plurality of secondary wound cavity tube members are perforated, share a common open pump-side end, and have distinct open wound-side ends, such that bodily fluids can enter the primary and secondary wound cavity tube members through the perforations and open wound-side ends;
wherein the open wound-side end of the solid outer tube is configured to attach to and form an airtight seal with the open pump-side end of the primary wound cavity tube;
wherein, when the open wound-side end of the solid outer tube member and the open pump-side end of the primary wound cavity tube are attached, the vacuum is operable to remove bodily fluids from throughout the branched tubing member via the negative pressure gradient;

3. The apparatus of claim 2, wherein when the vacuum and battery-powered pump are both activated, bodily fluids are actively drawn into the branched tubing member and drained out of the patient wound cavity via the solid outer tube, and the anticoagulant drug is delivered from the perforated inner tube to the solid outer tube via the plurality of perforations such that the anticoagulant drug does not enter any portion of the branched tubing member.

4. The apparatus of claim 3, wherein both the primary wound cavity tube and the plurality of secondary would cavity tube members of the branched tubing member are perforated and have closed wound-side ends, such that bodily fluids can enter the primary and secondary wound cavity tube members through the perforations.

5. The apparatus of claim 3, wherein both the primary wound cavity tube and the plurality of secondary wound cavity tube members of the branched tubing member are solid-walled and have open wound-side ends, such that bodily fluids can enter the primary and secondary wound cavity tube members through the open wound-side ends.

6. The apparatus of claim 3, wherein the primary wound cavity tube and the plurality of secondary cavity tube members of the branched tubing member are integrally formed.

7. The apparatus of claim 3, wherein the solid outer tube of the double-lumen tubing member and the primary wound cavity tube of the branched tubing member are integrally formed.

8. The apparatus of claim 3, wherein the solid outer tube of the double-lumen tubing member, the primary wound cavity tube, and the plurality of secondary cavity tube members are integrally formed.

9. The apparatus of claim 3, further comprising:
a one-way valve;
wherein the open wound-side end of the solid outer tube is attached to a first end of the one-way valve and the open pump-side end of the primary wound cavity tube is attached to a second end of the one-way valve, such that the one-way valve establishes an airtight one-way fluid connection between the two tubes and restricts fluid flow, only allowing fluid to flow in a direction away from the wound.

10. The apparatus of claim 3, wherein the branched tubing member is made of silicone.

11. A method of draining fluids from a wound, comprising the steps of:
Providing a double-lumen tubing member that comprises:
a perforated inner tube having a plurality of perforations for drug delivery; and
a solid outer tube for fluid drainage;
wherein the perforated inner tube and the solid outer tube are substantially concentric, each having a similarly located wound-side end and a distinct pump-side end;
wherein the wound-side end of the perforated inner tube is closed and the wound-side end of the solid outer tube is open;
Providing an anticoagulant drug;
Creating a fluid connection between the pump-side end of the perforated inner tube and a battery-powered pump that is operable to produce a positive pressure gradient and supply the anticoagulant drug through the length of the perforated inner tube;
Creating a fluid connection between the pump-side end of the solid outer tube and a vacuum that is operable to produce a negative pressure gradient and drain bodily fluids from inside of the outer tube member;

12. The method of claim 11, further comprising:
Providing a branched tubing member comprising:
a primary wound cavity tube; and
a plurality of secondary wound cavity tube members branching off from the primary wound cavity tube;
wherein each of the primary would cavity tube member and plurality of secondary wound cavity tube members have a wound-side end and a pump-side end;
wherein the primary wound cavity tube and the plurality of secondary wound cavity tube members are perforated, share a common open pump-side end, and have distinct open wound-side ends, such that bodily fluids can enter the primary and secondary wound cavity tube members through the perforations and open wound-side ends;
Placing the branched tubing member inside of a patient's wound cavity and sealing the branched tubing member therein;
Attaching the open wound-side end of the solid outer tube to the open pump-side end of the primary wound cavity tube such that an airtight seal is created between the two ends;
wherein, when the open wound-side end of the solid outer tube and the open pump-side end of the primary wound cavity tube are attached, the vacuum is operable to remove bodily fluids from throughout the branched tubing member via the negative pressure gradient;

13. The method of claim 12, further comprising:
Activating the vacuum to produce a negative pressure gradient and drain bodily fluids away from the patient wound cavity via the branched tubing member and the double-lumen tubing member;
Activating the battery-powered pump to produce positive pressure gradient and deliver the anticoagulant drug to the solid outer tube via the plurality of perforations in the perforated inner tube, such that the anticoagulant drug does not enter any portion of the branched tubing member.

14. The method of claim 13, wherein both the primary wound cavity tube and the plurality of secondary would cavity tube members of the branched tubing member are perforated and have closed wound-side ends, such that bodily fluids can enter the primary and secondary wound cavity tube members through the perforations thereon.

15. The method of claim 13, wherein the primary wound cavity tube and the plurality of secondary wound cavity tube members of the branched tubing member are solid-walled and have open wound-side ends, such that bodily fluids can enter the primary and secondary wound cavity tube members through their respective open wound-side ends.

16. The method of claim 13, wherein the primary wound cavity tube and the plurality of secondary cavity tube members of the branched tubing member are integrally formed.

17. The method of claim 13, wherein the solid outer tube of the double-lumen tubing member and the primary wound cavity tube of the branched tubing member are integrally formed.

18. The method of claim 13, wherein the solid outer tube of the double-lumen tubing member, the primary wound cavity tube, and the plurality of secondary cavity tube members are integrally formed.

19. The method of claim 13, further comprising:
Attaching the open wound-side end of the solid outer tube to a first end of a one-way valve;
Attaching the open pump-side end of the primary wound cavity tube to a second end of the one-way valve, such that the valve establishes an airtight one-way fluid connection between the two tubes and restricts fluid flow, only allowing fluid to flow in a direction away from the wound.

20. The method of claim 13, wherein the branched tubing member is made of silicone.
